(19) United States
(12) Patent Application Publication
(10) Pub. No.: US 2022/0144746 A1
(43) Pub. Date: May 12, 2022

(54) PROCESS FOR PRODUCING 2,2-DIALKYL-3-ACYLOXYPROPANALS

(71) Applicant: SIKA TECHNOLOGY AG, Baar (CH)

(72) Inventors: Urs BURCKHARDT, Zürich (CH); Andreas KRAMER, Zürich (CH); Michael GEYER, Schaffhausen (CH); John BARRATT, Manchester (GB)

(73) Assignee: SIKA TECHNOLOGY AG, Baar (CH)

(21) Appl. No.: 17/605,681

(22) PCT Filed: May 14, 2020
(86) PCT No.: PCT/EP2020/063538

§ 371 Date: Oct. 22, 2021

(30) Foreign Application Priority Data
May 17, 2019 (EP) 19175218.7

(51) Int. Cl. C07C 67/08; C07C 251/06; C07C 45/75; C08G 18/32

(52) U.S. Cl. CPC C07C 67/08; C08G 18/3256; C07C 45/75; C07C 251/06

(57) ABSTRACT

A process for preparing an aldol ester of the formula (I), wherein at least one carboxylic anhydride of the formula (II) is reacted with at least one aldol of the formula (III) with heating in the presence of a basic catalyst with a pKa of the conjugate acid of at least 8, and the reaction product obtained from the process. The process provides a light-coloured and low-odour reaction product which has a high content of aldol ester content of the formula (I) and can be used, without elaborate purification steps, particularly without overhead distillation of the aldol ester, as a blocking agent for primary amines. The resultant blocked amines have a long storage life together with polymers containing isocyanate groups, and cure rapidly and reliably on contact with moisture to form mechanically high-grade and robust elastomers.

### TECHNICAL FIELD

Production of aldol ester aldehydes and aldol ester aldimines and moisture-curing polyurethane compositions comprising said compounds, in particular for use as adhesive, sealant or coating.

### STATE OF THE ART

2,2-Dialkyl-3-acyloxypropanals are carboxylic esters of aldols from the crossed aldol reaction of secondary aliphatic aldehydes with formaldehyde. They are versatile starting materials for the production of, for example, fragrances, dyes, and polymers. Of particular commercial interest is the use thereof as blocking agents for primary polyamines. The aldol ester aldimines thereby obtained are particularly suitable as latent curing agents for polymers containing isocyanate groups. They afford polyurethane compositions having good storage stability that on contact with moisture cure quickly and with good process reliability to form stable elastomers of high mechanical quality, as are described by way of example in EP 1 527 115 or WO 2016/005457.

The preparation of 2,2-dialkyl-3-acyloxypropanals has been described many times in the literature. In the known methods of preparation, the aldol 2,2-dialkyl-3-hydroxypropanal is used either as is or is generated in situ from the starting aldehydes and esterified with a carboxylic acid, less commonly also with an anhydride or enol ester thereof, to form the aldol ester 2,2-dialkyl-3-acyloxypropanal. The esterification and also the concomitant aldol formation is typically carried out in the presence of acid catalysts such as sulfuric acid or p-toluenesulfonic acid and the aldol ester subsequently isolated and purified, in particular by distillation, as described for example in U.S. Pat. No. 3,251,876, 3,374,267 or 3,720,705.

The disadvantages of the described methods of preparation are that in practice they afford relatively low product yields. The reaction product thus obtained is typically very dark in color, with a pungent odor of strongly odorous by-products, and needs to be purified before it can be used further. Moreover, experience shows that the production process, for short-chain aldol esters in particular, under acid catalysis carries thermal process risks that make safe operation in a large-scale production facility impossible. This applies both to the reaction itself, even when this is operated without a solvent or entraining agent that limits the reaction temperature, and to the purification of the reaction product after the reaction, in particular by overhead distillation. For instance, at a temperature in the region of 150° C., strongly exothermic decomposition reactions already occur that cannot be adequately suppressed even through subsequent neutralization of the acid catalyst. In addition, the strongly acidic conditions make it necessary for production to be carried out in corrosion-proof facilities. Although the method of preparation without catalyst under neutral conditions described in U.S. Pat. No. 4,017,537 and the method of preparation with pyridine as catalyst described in DE 19 506728 give rise to no thermal process risks and no problems with corrosion, they are likewise unsatisfactory on account of the very long reaction times and relatively low yields obtained.

### SUMMARY OF THE INVENTION

It is therefore an object of the present invention to provide a method for preparing 2,2-dialkyl-3-acyloxypropanals that affords a high product yield and can be executed with good space-time yield without thermal process risks.

This object is achieved by the method as described in claim 1. In this method, a carboxylic anhydride is reacted with an aldol while heating in the presence of a basic catalyst having a conjugate acid pKa of at least 8. There has been no description to date of a method of this kind using a basic catalyst. It has surprisingly been found that the method of the invention makes possible a rapid reaction in high yield without thermal process risks and without the need for a solvent or an entraining agent. The reaction product obtained is surprisingly light in color and low in odor and can thus also be used without laborious purification, in particular without overhead distillation, in particular as a blocking agent for primary amines. Because there is no corrosion effect on metals, the method of the invention can be executed in inexpensive standard reactors made of stainless steel. A particular surprise with the method of the invention is that the reaction product is stable on heating to well over 200° C. even in the case of short-chain aldol esters, in particular 2,2-dialkyl-3-acetyloxypropanals, whereas heating the corresponding reaction products from acid-catalyzed processes to above 150° C. results in the observation of strong exothermicity indicative of an appreciable thermal process risk.

The method of the invention affords a reaction product that is light in color and low in odor and with a high content of 2,2-dialkyl-3-acyloxypropanal, which can be used without laborious purification steps, in particular without overhead distillation of the 2,2-dialkyl-3-acyloxypropanal, as a blocking agent for primary amines. The blocked amines/latent curing agents thereby obtained are low in odor, are surprisingly storage-stable in combination with polymers containing isocyanate groups, and on contact with moisture cure quickly and with good process reliability to form stable elastomers of high mechanical quality,

Further aspects of the invention are the subject of further independent claims. Particularly preferred embodiments of the invention are the subject of the dependent claims.

### WAYS OF EXECUTING THE INVENTION

The invention provides a method for preparing an aldol ester of the formula (I),

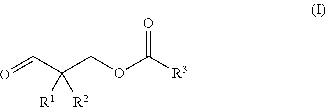

where R¹ and R² are identical or different alkyl radicals having 1 to 4 carbon atoms or together are an alkylene radical having 4 to 6 carbon atoms, and R³ is an optionally halogenated hydrocarbyl radical having 1 to 17 carbon atoms,
   characterized in that at least one carboxylic anhydride of the formula (II)

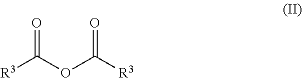

is reacted with at least one aldol of the formula (III),

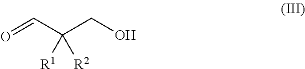

optionally in the form of an oligomer thereof, while heating in the presence of a basic catalyst having a conjugate acid pKa of at least 8.

An “aliphatic” aldehyde group or isocyanate group refers to one that is attached directly to an aliphatic or cycloaliphatic carbon atom.

An “aromatic” aldehyde group or isocyanate group refers to one that is attached directly to an aromatic carbon atom.

A “primary amino group” refers to an amino group that is attached to a single organic radical and bears two hydrogen atoms; a “secondary amino group” refers to an amino group that is attached to two organic radicals, which may also together be part of a ring, and bears one hydrogen atom; and a “tertiary amino group” refers to an amino group that is attached to three organic radicals, two or three of which may also be part of one or more rings, and does not bear any hydrogen atoms.

Substance names beginning with “poly”, such as polyamine, polyol or polyisocyanate, refer to substances that formally contain two or more of the functional groups that occur in their name per molecule.

“Molecular weight” refers to the molar mass (in g/mol) of a molecule or a molecule residue. “Average molecular weight” refers to the number-average molecular weight (Mₙ) of a polydisperse mixture of oligomeric or polymeric molecules or molecule residues. It is determined by gel-permeation chromatography (GPC) against polystyrene as standard.

Percent by weight (% by weight) values refer to the proportions by mass of a constituent in a composition based on the overall composition, unless otherwise stated. The terms “mass” and “weight” are used synonymously in the present document.

“NCO content” refers to the content of isocyanate groups in % by weight.

A substance or composition is referred to as “storage-stable” or “storable” when it can be stored at room temperature in a suitable container for a prolonged period, typically for at least 3 months up to 6 months or longer, without this storage resulting in any change in its application or use properties to an extent relevant to its use.

“Room temperature” refers to a temperature of 23° C.

All industry standards and norms mentioned in this document relate to the versions valid at the date of first filing.

Preferably, R¹ is methyl or ethyl, in particular methyl, and R² is methyl, ethyl, n-propyl or n-butyl.

More preferably, R¹ and R² are each methyl.

Preferably, R³ is an optionally chlorinated hydrocarbyl radical having 1 to 11 carbon atoms.

More preferably, R³ is an alkyl radical having 1 to 7 carbon atoms or is phenyl. Most preferably, R³ is methyl.

The preferred radicals R¹, R², and R³ are particularly easily obtainable and afford aldol esters of the formula (I), which are particularly suitable as blocking agents for primary amines.

In the case of small radicals R³, in particular methyl, the method of the invention is particularly advantageous, since the known acid-catalyzed methods of the prior art give rise to intensely colored, strongly odorous, and thermally unstable reaction products with high process risk. Blocked amines based on aldol esters of the formula (I) that have small radicals R³, in particular methyl, are particularly suitable for moisture-curing polyurethane compositions that need to have particularly low viscosity and/or particularly high hardness, for example for coatings.

The basic catalyst preferably has a conjugate acid pKa of at least 9, in particular at least 10. This achieves a particularly rapid reaction.

Preferably, the basic catalyst is a tertiary amine or an amidine.

More preferably, the basic catalyst is selected from the group consisting of trimethylamine, dimethylethylamine, methyldiethylamine, triethylamine, diisopropylethylamine, N-methylpyrrolidine, N-methylpiperidine, 1,5-diazabicyclo[4.3.0]non-5-ene (DBN), and 1,8-diazabicyclo[5.4.0]undec-7-ene (DBU). These compounds are easily accessible and exhibit good catalytic activity in the method of the invention.

Most preferred is triethylamine. This makes possible a particularly rapid reaction, is inexpensive, and is volatile and can thus be readily removed from the reaction mixture by distillation. It is also of excellent suitability as catalyst for the preceding preparation of the aldol of the formula (III).

The basic catalyst is preferably used in an amount within a range from 0.01% to 10% by weight, in particular 0.05% to 5% by weight, based on the total reaction mixture.

The triethylamine that is the most preferred catalyst is preferably used in an amount within a range from 0.1% to 10% by weight, in particular 0.5% to 5% by weight, based on the total reaction mixture.

The method is preferably executed at a temperature within a range from 80 to 150° C., in particular 100 to 130° C.

Preferably, the carboxylic anhydride of the formula (II) is used in a stoichiometric excess in relation to the aldol of the formula (III).

Preferably, the aldol of the formula (III) is initially charged and the carboxylic anhydride of the formula (II) added in the presence of the basic catalyst.

The carboxylic acid liberated from the carboxylic anhydride, unreacted carboxylic anhydride, the basic catalyst, and any volatile by-products and solvents present are preferably largely or completely removed from the reaction mixture during or after the reaction, in particular by distillation under reduced pressure.

Optionally, a solvent or entraining agent may be used, in particular cyclohexane or toluene or a hydrocarbon mixture such as petroleum spirit or hydrotreated naphtha light, in particular having a boiling range of from 75 to 95° C. or 80 to 100° C.

The method is preferably executed without using an organic solvent or entraining agent.

The carboxylic anhydride of the formula (II) is preferably selected from the group consisting of acetic anhydride, propionic anhydride, butyric anhydride, isobutyric anhydride, hexanoic anhydride, 2-ethylhexanoic anhydride, lauric anhydride, benzoic anhydride, chloroacetic anhydride, dichloroacetic anhydride, and trichloroacetic anhydride.

Preference among these is given to acetic anhydride, propionic anhydride, hexanoic anhydride, 2-ethylhexanoic anhydride or benzoic anhydride

Most preferred is acetic anhydride.

The aldol of the formula (III) is optionally used in the form of an oligomer, in particular in the form of a dimer of the formula (IIIa).

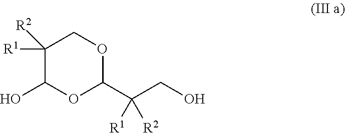

The aldol of the formula (III) or an oligomer thereof is preferably obtained from the reaction of formaldehyde, optionally in the form of paraformaldehyde or trioxane, with an aldehyde of the formula (IV),

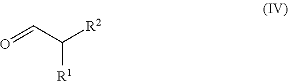

where R¹ and R² are as defined previously.

Formaldehyde is preferably used as formalin or in the form of paraformaldehyde, more preferably in the form of paraformaldehyde.

The aldehyde of the formula (IV), is preferably isobutyraldehyde, 2-methylbutyraldehyde, 2-ethylbutyraldehyde, 2-methylvaleraldehyde or 2-ethylcaproaldehyde.

Particularly preferred is isobutyraldehyde.

The aldol of the formula (III) is preferably used as constituent of a reaction mixture obtained from the reaction of formaldehyde, optionally in the form of paraformaldehyde or trioxane, with at least one aldehyde of the formula (IV),

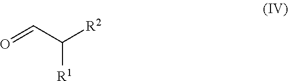

in the presence of a basic catalyst having a conjugate acid pKa of at least 8.

This reaction mixture containing the aldol of the formula (III) is in particular free of strong acids, in particular halogen-containing acids such as boron trichloride, boron tribromide or hydrochloric acid. This means that the basic catalyst does not give rise to any salt formation, which would interfere with its activity.

The reaction of formaldehyde with at least one aldehyde of the formula (IV) is a crossed aldol reaction. It is preferably carried out in the presence of a basic catalyst having a conjugate acid pKa of at least 8, preferably of at least 9, in particular of at least 10. It is preferably the same basic catalyst as is used in the esterification reaction of the carboxylic anhydride of the formula (II) with the aldol of the formula (III), i.e. in the method of the invention for preparing an aldol ester of the formula (I). The basic catalyst for both reactions is particularly preferably triethylamine.

The basic catalyst for the aldol reaction is preferably used in an amount within a range from 0.1% to 20% by weight, in particular 0.5% to 15% by weight, based on the total reaction mixture for the aldol reaction.

The aldol reaction is preferably carried out at a temperature within a range from 60 to 90° C.

The aldehyde of the formula (IV) is preferably used in a stoichiometric excess in relation to formaldehyde.

Formaldehyde is preferably used as formalin or in the form of paraformaldehyde, in particular in the form of paraformaldehyde.

In the aldol reaction there may be a solvent present. Preferably, the aldol reaction is carried out without organic solvent.

The aldol reaction is preferably followed by the removal of volatiles from the reaction mixture, in particular of unreacted aldehyde of the formula (IV), solvents, and optionally part of the basic catalyst, in particular by distillation under reduced pressure.

The method of the invention is particularly preferably executed in two stages, wherein
(i) in the first step (aldol reaction), the basic catalyst and formaldehyde, in particular in the form of paraformaldehyde, are initially charged, then at least one aldehyde of the formula (IV) is added in stoichiometric excess in relation to formaldehyde at a temperature within a range from 60 to 90° C., resulting in the formation of the aldol of the formula (III), after which volatiles, in particular excess aldehyde of the formula (IV) and optionally part of the basic catalyst, are removed from the reaction mixture, and
(ii) in the second step (esterification), the reaction mixture thus obtained is reacted with the carboxylic anhydride of the formula (II) at a temperature within a range from 100 to 130° C., wherein volatiles, in particular carboxylic acid liberated from the carboxylic anhydride, unreacted carboxylic anhydride, and optionally the basic catalyst, are removed from the reaction mixture during and/or after the reaction, in particular by distillation under reduced pressure.

The invention further provides the reaction product obtained from the method of the invention, in particular the reaction product obtained from the preferred two-stage method, characterized in that it comprises 60% to 95% by weight, in particular 65% to 90% by weight, more preferably 70% to 85% by weight, of aldol ester of the formula (I) and 5% to 40% by weight, preferably 10% to 35% by weight, in particular 15% to 30% by weight, of other esters, aldehydes and/or acetals not corresponding to the formula (I).

The aldol ester of the formula (I) present in the reaction product is preferably selected from the group consisting of 2,2-dimethyl-3-acetoxypropanal, 2,2-dimethyl-3-propionoxypropanal, 2,2-dimethyl-3-hexanoyloxypropanal, 2,2-dimethyl-3-(2-ethylhexanoyloxy)propanal, and 2,2-dimethyl-3-benzoyloxypropanal. Particular preference is given to 2,2-dimethyl-3-acetoxypropanal.

In addition to the aldol ester of the formula (I), the reaction product of the invention preferably comprises triesters of the formula (V) and/or acetals of the formula (VI).

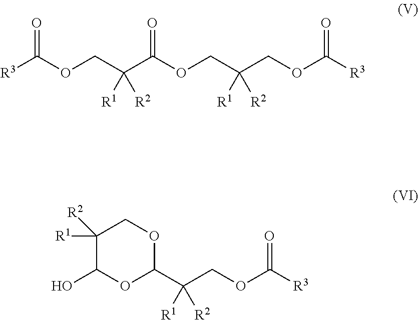

R¹, R², and R³ in formulas (V) and (VI) are as defined previously.

The reaction product of the invention preferably comprises 0.1% to 20% by weight, in particular 0.5% to 15% by weight, more preferably 1% to 10% by weight, of triesters of the formula (V).

The reaction product of the invention preferably comprises 1% to 20% by weight, in particular 2% to 15% by weight, more preferably 3% to 10% by weight, of acetals of the formula (VI).

The reaction product of the invention has the advantage that it is free of halides and thus does not need to be freed from them through laborious workup processes.

The reaction product of the invention is clear, light in color, and low in odor. It can accordingly be used even without further purification. The reaction product is thermally very stable and shows no appreciable exothermicity on heating to 200° C. This enables high process safety in the preparation and processing thereof.

The reaction product of the invention can be purified further before use for isolation of the aldol ester of the formula (I), in particular by overhead distillation. The high thermal stability of the reaction product is particularly advantageous here.

The reaction product of the invention is preferably used without further purification.

The reaction product of the invention is suitable for a large number of uses, in particular for the production of fragrances, dyes or polymers. The reaction product of the invention is particularly suitable as a blocking agent for primary amines.

Preference is given to using the reaction product of the invention for the production of blocked amines. For this, the reaction product is reacted with at least one primary amine. In the reaction, the primary amino groups react with the aldehyde groups in a condensation reaction that results in the liberation of water and the formation of aldimine groups, which represent a blocked, hydrolytically activatable form of the primary amino groups.

The blocked amines obtained from the reaction of the reaction product of the invention with primary amines can be used advantageously as latent curing agents in moisture-curing polyurethane compositions.

For the reaction with the reaction product of the invention, preference is given to primary amines that are difunctional with respect to isocyanate groups, i.e. primary amines that in addition to a primary amino group also have at least one further primary amino group and/or at least one secondary amino group and/or at least one hydroxyl group. The blocked amines thereby obtained are particularly suitable as latent curing agents for polyurethane compositions. These have particularly advantageous properties in relation to storage stability, processability, curing, and mechanical properties.

The invention thus further provides a blocked amine obtained from reacting the reaction product of the invention with at least one amine that has a primary amino group and additionally at least one reactive group selected from primary amino group, secondary amino group, and hydroxyl group. Preferably, the amine contains only a secondary amino group or only a hydroxyl group. Particularly preferably, the amine is free of secondary amino groups.

A blocked amine thus obtained contains, in addition to the aldimine from the reaction of the aldol ester of the formula (I), the by-products from the method of the invention present in the reaction product used, in particular the described triesters of the formula (V) and/or acetals of the formula (VI) and/or reaction products thereof with the amine.

Suitable amines for blocking are in particular
primary aliphatic diamines, such as in particular ethane-1,2-diamine, propane-1,2-diamine, propane-1,3-diamine, butane-1,4-diamine, butane-1,3-diamine, 2-methylpropane-1,2-diamine, pentane-1,3-diamine, pentane-1,5-diamine, 2,2-dimethylpropane-1,3-diamine, hexane-1,6-diamine, 1,5-diamino-2-methylpentane, heptane-1,7-diamine, octane-1,8-diamine, 2,5-dimethylhexane-1,6-diamine, nonane-1,9-diamine, 2,2(4),4-trimethylhexane-1,6-diamine, decane-1,10-diamine, undecane-1,11-diamine, 2-butyl-2-ethylpentane-1,5-diamine, dodecane-1,12-diamine, cyclohexane-1,2-diamine, cyclohexane-1,3-diamine, cyclohexane-1,4-diamine, 1-amino-3-aminomethyl-3,5,5-trimethylcyclohexane, 4(2)-methylcyclohexane-1,3-diamine, 1,3-bis(aminomethyl)cyclohexane, 1,4-bis(aminomethyl)cyclohexane, bis(4-aminocyclohexyl)methane, bis(4-amino-3-methylcyclohexyl)methane, bis(4-amino-3-ethylcyclohexyl)methane, bis(4-amino-3,5-dimethylcyclohexyl)methane, bis(4-amino-3-ethyl-5-methylcyclohexyl)methane, 2,5(2,6)-bis(aminomethyl)bicyclo[2.2.1]heptane, 3(4),8(9)-bis(aminomethyl)tricyclo[5.2.1.0^{2,6}]decane, 1,3-bis(aminomethyl)benzene, 1,4-bis(aminomethyl)benzene, 3-oxapentane-1,5-diamine, 3,6-dioxaoctane-1,8-diamine, 4,7-dioxadecane-1,10-diamine, 4,7-dioxadecane-2,9-diamine, 4,9-dioxadodecane-1,12-diamine, 5,8-dioxadodecane-3,10-diamine, 4,7,10-trioxatridecane-1,13-diamine, am-polyoxypropylenediamines having an average molecular weight Mₙ within a range from 170 to 4000 g/mol, in particular the Jeffamine® products D-230, D-400, XTJ-582, D-2000, XTJ-578 or D-4000 (all from Huntsman), α,ω-polyoxypropylene/polyoxyethylenediamine, in particular the Jeffamine® products ED-600, ED-900, ED-2003 or HK-511 (all from Huntsman), α,ω-polyoxypropylene/polyoxy-1,4-butylenediamine, in particular the Jeffamine® products THF-100, THF-140, THF-230, XTJ-533 or XTJ-536 (all from Huntsman), α,ω-polyoxypropylene/polyoxy-1,2-butylenediamine, in particular the Jeffamine® products XTJ-568 or XTJ-569 (both from Huntsman) or α,ω-polyoxy-1,2-butylenediamine, in particular Jeffamine® XTJ-523 (from Huntsman),
primary aliphatic triamines, such as in particular 1,3,6-triaminohexane, 1,4,8-triaminooctane, 4-aminomethyloctane-1,8-diamine, 5-aminomethyloctane-1,8-diamine, 1,6,11-triaminoundecane, 1,3,5-triaminocyclohexane, 1,3,5-tris(aminomethyl)cyclohexane, 1,3,5-tris(aminomethyl)benzene, trimethylolpropane- or glycerol-started tris(ω-polyoxypropylenamine) having an average molecular weight Mₙ within a range from 330 to 6000 g/mol, in particular the Jeffamine® products T-403, T-3000 or T-5000 (all from Huntsman), or trimethylolpropane-started tris(ω-polyoxypropylene/polyoxy-1,2-butylenamine), in particular Jeffamine® XTJ-566 (from Huntsman), or
primary aromatic diamines such as in particular 1,3-phenylenediamine, 1,4-phenylenediamine, 4(2)-methyl-1,3-phenylenediamine (TDA), 3,5-diethyl-2,4(6)-tolylenediamine (DETDA) or 4,4′-diaminodiphenylmethane (MDA), or
aliphatic diamines having a primary and a secondary amino group, such as in particular N-methylethane-1,2-diamine, N-ethylethane-1,2-diamine, N-butylethane-1,2-diamine, N-hexylethane-1,2-diamine, N-(2-ethylhexyl)ethane-1,2-diamine, N-cyclohexylethane-1,2-diamine, N-benzylethane-1,2-diamine, 4-aminomethylpiperidine, 3-(4-aminobutyl)piperidine, N-(2-aminoethyl)piperazine, N-(2-aminopropyl)piperazine, N-benzylpropane-1,2-diamine, N-benzylpropane-1,3-diamine, N-methylpropane-1,3-diamine, N-ethylpropane-1,3-diamine, N-butylpropane-1,3-diamine, N-hexylpropane-1,3-diamine, N-(2-ethylhexyl)propane-1,3-diamine, N-dodecylpropane-1,3-diamine, N-cyclohexylpropane-1,3-diamine, 3-methylamino-1-pentylamine, 3-ethylamino-1-pentylamine, 3-butylamino-1-pentylamine, 3-hexylamino-1-pentylamine, 3-(2-ethylhexyl)amino-1-pentylamine, 3-dodecylamino-1-pentylamine, 3-cyclohexylamino-1-pentylamine, fatty diamines, such as N-cocoalkylpropane-1,3-diamine, N-oleylpropane-1,3-diamine, N-soyaalkylpropane-1,3-diamine, N-tallowalkylpropane-1,3-diamine or N—(C₁₆₋₂₂ alkyl)propane-1,3-diamine, such as are available for example under the Duomeen® trade name from Akzo Nobel, or products from the Michael-type addition of aliphatic primary diamines to acrylonitrile, maleic or fumaric diesters, citraconic diesters, (meth)acrylic esters, (meth)acrylamides or itaconic diesters, reacted in the molar ratio of 1:1, or
aliphatic polyamines having two primary and a secondary amino group, such as in particular bis(hexamethylene)triamine (BHMT), diethylenetriamine (DETA), dipropylenetriamine (DPTA), N-(2-aminoethyl)propane-1,3-diamine (N3 amine), N3-(3-aminopentyl)pentane-1,3-diamine or N5-(3-amino-1-ethylpropyl)-2-methylpentane-1,5-diamine, or
hydroxylamines, such as in particular 2-aminoethanol, 2-amino-1-propanol, 1-amino-2-propanol, 3-amino-1-propanol, 4-amino-1-butanol, 4-amino-2-butanol, 2-amino-2-methylpropanol, 5-amino-1-pentanol, 6-amino-1-hexanol, 7-amino-1-heptanol, 8-amino-1-octanol, 10-amino-1-decanol, 12-amino-1-dodecanol or higher homologs thereof, 4-(2-aminoethyl)-2-hydroxyethylbenzene, 3-aminomethyl-3,5,5-trimethylcyclohexanol, derivatives bearing a primary amino group of glycols such as diethylene glycol, dipropylene glycol, dibutylene glycol or higher oligomers or polymers of these glycols, in particular 2-(2-aminoethoxy)ethanol, 2-(2-(2-aminoethoxy)ethoxy)ethanol or α-(2-hydroxymethylethyl)-ω-(2-aminomethylethoxy)-poly(oxy(methylethane-1,2-diyl), 3-(2-hydroxyethoxy)propylamine, 3-(2-(2-hydroxyethoxy)ethoxy)propylamine or 3-(6-hydroxyhexyloxy)propylamine.

The amine is in particular selected from the group consisting of hexane-1,6-diamine, 1-amino-3-aminomethyl-3,5,5-trimethylcyclohexane, 4(2)-methylcyclohexane-1,3-diamine, 1,3-bis(aminomethyl)cyclohexane, 1,4-bis(aminomethyl)cyclohexane, 1,3-bis(aminomethyl)benzene, cyclohexane-1,2-diamine, cyclohexane-1,3-diamine, cyclohexane-1,4-diamine, bis(4-aminocyclohexyl)methane, 2,5(2,6)-bis(aminomethyl)bicyclo[2.2.1]heptane, 3(4),8(9)-bis(aminomethyl)tricyclo[5.2.1.0^{2,6}]decane, α,ω-polyoxypropylenediamine having an average molecular weight Mₙ within a range from 170 to 500 g/mol, in particular the Jeffamine® products D-230 or D-400 (from Huntsman), trimethylolpropane- or glycerol-started tris(ω-polyoxypropylenamine) having an average molecular weight Mₙ within a range from 330 to 500 g/mol, in particular Jeffamine® T-403 (from Huntsman), 1,4-phenylenediamine, 3,5-diethyl-2,4(6)-tolylenediamine, 2-(2-aminoethoxy)ethanol, 2-(2-(2-aminoethoxy)ethoxy)ethanol, and 3-aminomethyl-3,5,5-trimethylcyclohexanol.

Preference among these is given to hexane-1,6-diamine, 1-amino-3-aminomethyl-3,5,5-trimethylcyclohexane, α,ω-polyoxypropylenediamine having an average molecular weight Mₙ within a range from 170 to 300 g/mol, trimethylolpropane-started tris(ω-polyoxypropyleneamine) having an average molecular weight Mₙ within a range from 330 to 500 g/mol or 2-(2-aminoethoxy)ethanol.

The preferred amines are easily obtainable. In blocked form, they afford moisture-curing polyurethane compositions having good storage stability, good processability, rapid curing, and high strength coupled with high extensibility. If the blocked amine has a hydroxyl group or a secondary amino group, this group during storage reacts with isocyanate groups that are present.

The blocked amine of the invention is preferably prepared by
combining the reaction product of the invention with the amine into a reaction mixture, optionally with addition of a solvent, wherein the aldehyde groups are present stoichiometrically or in a stoichiometric excess in relation to the primary amino groups, and
removing from the reaction mixture during or after said combining, by means of a suitable method, the water of condensation formed in the reaction and any solvent optionally used.

The water of condensation and any solvent optionally used are preferably removed from the heated reaction mixture by application of reduced pressure.

Preferably, no solvent is used.

The reaction is preferably carried out at a temperature within a range from 20° C. to 120° C., in particular 40° C. to 100° C.

A catalyst is optionally used in the reaction, in particular an acid catalyst.

The blocked amine of the invention comprises in particular at least one aldimine of the formula (VII),

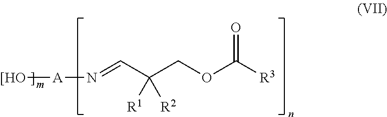

where
m is 0 or 1, n is 1 or 2 or 3, and (m+n) is 2 or 3,
A is a (m+n)-valent organic radical having 2 to 25 carbon atoms, and
R¹, R² and R³ are as defined above.

Preferably, m is 0 and n is 2 or 3. Such an aldimine of formula (VII) is a di- or trialdimine.

More preferably, m is 1 and n is 1. Such an aldimine of formula (VII) is a hydroxyaldimine.

A is preferably an alkylene radical optionally having cyclic components or a di- or trivalent polyoxyalkylene radical having 5 to 15 carbon atoms.

A is particularly preferably a radical selected from the group consisting of 1,6-hexylene, (1,5,5-trimethylcyclohexan-1-yl)methane-1,3, α,ω-polyoxypropylene having an average molecular weight Mₙ within a range from 170 to 300 g/mol, trimethylolpropane-started tris(ω-polyoxypropylene) having an average molecular weight Mₙ within a range from 330 to 500 g/mol, 1,4-phenylene, 3,5-diethyl-2,4(6)-tolylene, and 3-oxa-1,5-pentylene.

The aldimine of the formula (VII) is particularly preferably selected from the group consisting of N,N′-bis(2,2-dimethyl-3-acetoxypropylidene)hexylene-1,6-diamine, N, N′-bis(2,2-dimethyl-3-acetoxypropylidene)-3-aminomethyl-3,5,5-trimethylcyclohexylamine, N,N′-bis(2,2-dimethyl-3-acetoxypropylidene)polyoxypropylendiamine having an average molecular weight Mₙ within a range from 450 to 880 g/mol, N,N′,N″-tris(2,2-dimethyl-3-acetoxypropylidene)polyoxypropylentriamine having an average molecular weight Mₙ within a range from 730 to 880 g/mol, N,N′-bis(2,2-dimethyl-3-acetoxypropylidene)phenylene-1,4-diamine, N,N′-bis(2,2-dimethyl-3-acetoxypropylidene)-3,5-diethyl-tolylene-2,4(6)-diamine, and N-(2,2-dimethyl-3-acetoxypropylidene)-2-(2-aminoethoxy)ethan-1-ol.

The preferred blocked amines afford moisture-curing polyurethane compositions having good storage stability, good processability, particularly rapid curing, and particularly high strength coupled with high extensibility. In the case of N-(2,2-dimethyl-3-acetoxypropylidene)-2-(2-aminoethoxy)ethan-1-ol, the hydroxyl group during storage reacts with isocyanate groups that are present.

The invention further provides a moisture-curing polyurethane composition comprising
at least one polyisocyanate and/or polymer containing isocyanate groups and
at least one blocked amine from the reaction of the reaction product of the invention, as described above.

The moisture-curing polyurethane composition preferably comprises a blocked amine comprising at least one aldimine of the formula (VII).

Suitable polyisocyanates are
commercially available aromatic, aliphatic or cycloaliphatic diisocyanates, such as in particular diphenylmethane 4,4′-diisocyanate, optionally containing fractions of diphenylmethane 2,4′- and/or 2,2′-diisocyanate (MDI), diphenylmethane 2,4′-diisocyanate (2,4′-MDI), tolylene 2,4-diisocyanate or mixtures thereof with tolylene 2,6-diisocyanate (TDI), phenylene 1,4-diisocyanate (PDI), naphthalene 1,5-diisocyanate (NDI), hexane 1,6-diisocyanate (HDI), 2,2(4),4-trimethylhexamethylene 1,6-diisocyanate (TMDI), cyclohexane 1,3- or 1,4-diisocyanate, 1-isocyanato-3,3,5-trimethyl-5-isocyanatomethylcyclohexane (isophorone diisocyanate or IPDI), perhydrodiphenylmethane 2,4′- or 4,4′-diisocyanate (HMDI), 1,3- or 1,4-bis(isocyanatomethyl)cyclohexane, m- or p-xylylene diisocyanate (XDI), or mixtures thereof,
higher functional derivatives of such diisocyanates, in particular diphenylmethane 4,4′-diisocyanate liquefied through carbodiimidization or uretonimine formation or adduct formation with polyols,
a room temperature liquid mixture of MDI with MDI homologs (polymeric MDI or PMDI),
diisocyanate oligomers, such as in particular HDI biurets such as Desmodur® N 100 or N 3200 (from Covestro), Tolonate® HDB or HDB-LV (from Vencorex) or Duranate® 24A-100 (from Asahi Kasei); HDI isocyanurates such as Desmodur® N 3300, N 3600 or N 3790 BA (all from Covestro), Tolonate® HDT, HDT-LV or HDT-LV2 (from Vencorex), Duranate® TPA-100 or THA-100 (from Asahi Kasei) or Coronate® HX (from Tosoh Corp.); HDI uretdiones such as Desmodur® N 3400 (from Covestro); HDI iminooxadiazinediones such as Desmodur® XP 2410 (from Covestro); HDI allophanates such as Desmodur® VP LS 2102 (from Covestro); IPDI isocyanurates, for example in solution as Desmodur® Z 4470 (from Covestro) or in solid form as Vestanat® T1890/100 (from Evonik Industries); TDI oligomers such as Desmodur® IL (from Covestro); or mixed isocyanurates based on TDI/HDI, such as Desmodur® HL (from Covestro); or
commercially available triisocyanates, such as in particular 4,4′,4″-triphenylmethane triisocyanate, available as Desmodur® RE (from Covestro), or tris(p-isocyanatophenyl) thiophosphate, available as Desmodur® RFE (from Covestro).

A suitable polymer containing isocyanate groups is in particular a reaction product of at least one polyol with a superstoichiometric amount of at least one diisocyanate. The reaction is preferably carried out with exclusion of moisture at a temperature within a range from 20 to 160° C., in particular 40 to 140° C., optionally in the presence of suitable catalysts.

The NCO/OH ratio is preferably within a range from 1.3/1 to 10/1. The monomeric diisocyanate remaining in the reaction mixture after reaction of the OH groups can be removed, in particular by distillation.

If monomeric diisocyanate is removed from the polymer, the NCO/OH ratio in the reaction is preferably within a range from 3/1 to 10/1, in particular 4/1 to 7/1, and the resulting polymer containing isocyanate groups comprises after the distillation preferably not more than 0.5% by weight, more preferably not more than 0.3% by weight, of monomeric diisocyanate. Monomeric diisocyanate is in particular removed here by short-path distillation under reduced pressure.

If no monomeric diisocyanate is removed from the polymer, the NCO/OH ratio in the reaction is preferably within a range from 1.3/1 to 2.5/1. Such a polyether urethane polymer in particular comprises not more than 3% by weight, preferably not more than 2% by weight, of monomeric diisocyanate.

Preferred monomeric diisocyanates are the aromatic, aliphatic or cycloaliphatic diisocyanates already mentioned, in particular MDI, TDI, HDI, HMDI or IPDI, or mixtures thereof.

Particular preference is given to 4,4′-MDI, TDI or IPDI.

Suitable polyols are commercially available polyols or mixtures thereof, in particular
polyether polyols, in particular polyoxyalkylene diols and/or polyoxyalkylene triols, in particular polymerization products of ethylene oxide or 1,2-propylene oxide or 1,2- or 2,3-butylene oxide or oxetane or tetrahydrofuran or mixtures thereof, where these may be polymerized with the aid of a starter molecule having two or three active hydrogen atoms, in particular a starter molecule such as water, ammonia or a compound having two or more OH or NH groups, for example ethane-1,2-diol, propane-1,2- or -1,3-diol, neopentyl glycol, diethylene glycol, triethylene glycol, the isomeric dipropylene glycols or tripropylene glycols, the isomeric butanediols, pentanediols, hexanediols, heptanediols, octanediols, nonanediols, decanediols, undecanediols, cyclohexane-1,3- or -1,4-dimethanol, bisphenol A, hydrogenated bisphenol A, 1,1,1-trimethylolethane, 1,1,1-trimethylolpropane, glycerol or aniline, or mixtures of the abovementioned compounds. Likewise suitable are polyether polyols with polymer particles dispersed therein, in particular those with styrene/acrylonitrile (SAN) particles or polyurea or polyhydrazodicarbonamide (PHD) particles.

Preferred polyether polyols are polyoxypropylene diols or polyoxypropylene triols, or what are called ethylene oxide-terminated (EO-capped or EO-tipped) polyoxypropylene diols or triols. The latter are mixed polyoxyethylene/polyoxypropylene polyols that are in particular obtained when polyoxypropylene diols or triols, on conclusion of the polypropoxylation reaction, undergo further alkoxylation with ethylene oxide that results in them having primary hydroxyl groups.

Preferred polyether polyols have a degree of unsaturation of less than 0.02 meq/g, in particular less than 0.01 meq/g.
Polyester polyols, also called oligoesterols, prepared by known processes, in particular the polycondensation of hydroxycarboxylic acids or lactones or the polycondensation of aliphatic and/or aromatic polycarboxylic acids with di- or polyhydric alcohols. Preference is given to polyester diols from the reaction of dihydric alcohols, such as in particular ethane-1,2-diol, diethylene glycol, propane-1,2-diol, dipropylene glycol, butane-1,4-diol, pentane-1,5-diol, hexane-1,6-diol, neopentyl glycol, glycerol, 1,1,1-trimethylolpropane or mixtures of the abovementioned alcohols, with organic dicarboxylic acids or the anhydrides or esters thereof, such as in particular succinic acid, glutaric acid, adipic acid, suberic acid, sebacic acid, dodecanedicarboxylic acid, maleic acid, fumaric acid, phthalic acid, isophthalic acid, terephthalic acid, cyclohexane-1,2-dicarboxylic acid, cyclohexane-1,3-dicarboxylic acid or -1,4-dicarboxylic acid or mixtures of the abovementioned acids, or polyester polyols formed from lactones such as in particular ε-caprolactone. Particular preference is given to polyester polyols formed from adipic acid or sebacic acid or dodecanedicarboxylic acid and hexanediol or neopentyl glycol.
Polycarbonate polyols as obtainable by reaction, for example, of the abovementioned alcohols—used to form the polyester polyols—with dialkyl carbonates, diaryl carbonates or phosgene.
Block copolymers bearing at least two OH groups and having at least two different blocks having polyether, polyester and/or polycarbonate structure of the type described above, in particular polyether polyester polyols.
Polyacrylate or polymethacrylate polyols.
Polyhydroxy-functional fats or oils, for example natural fats and oils, in particular castor oil; or polyols obtained by chemical modification of natural fats and oils—called oleochemical polyols—for example the epoxy polyesters or epoxy polyethers obtained by epoxidation of unsaturated oils and subsequent ring opening with carboxylic acids or alcohols, or polyols obtained by hydroformylation and hydrogenation of unsaturated oils; or polyols obtained from natural fats and oils by breakdown processes such as alcoholysis or ozonolysis and subsequent chemical linkage, for example by transesterification or dimerization, of the breakdown products or derivatives thereof thus obtained. Suitable breakdown products of natural fats and oils are in particular fatty acids and fatty alcohols and also fatty acid esters, in particular the methyl esters (FAME), which can be derivatized to hydroxy fatty acid esters, for example by hydroformylation and hydrogenation.
Polyhydrocarbon polyols, also called oligohydrocarbonols, such as in particular polyhydroxy-functional polyolefins, polyisobutylenes, polyisoprenes; polyhydroxy-functional ethylene/propylene, ethylene/butylene or ethylene/propylene/diene copolymers, as produced for example by Kraton Polymers; polyhydroxy-functional polymers of dienes, in particular of 1,3-butadiene, which can in particular also be produced from anionic polymerization; polyhydroxy-functional copolymers of dienes, such as 1,3-butadiene, or diene mixtures and vinyl monomers, such as styrene, acrylonitrile, vinyl chloride, vinyl acetate, vinyl alcohol, isobutylene or isoprene, in particular polyhydroxy-functional acrylonitrile/butadiene copolymers, as can in particular be produced from epoxides or amino alcohols and carboxyl-terminated acrylonitrile/butadiene copolymers (commercially available for example under the Hypro® CTBN or CTBNX or ETBN name from Emerald Performance Materials); or hydrogenated polyhydroxy-functional polymers or copolymers of dienes.

Also especially suitable are mixtures of polyols.

Preference is given to polyether polyols, polyester polyols, polycarbonate polyols, poly(meth)acrylate polyols or polybutadiene polyols.

Particular preference is given to polyether polyols, polyester polyols, in particular aliphatic polyester polyols, or polycarbonate polyols, in particular aliphatic polycarbonate polyols.

Especially preferred are polyether polyols, in particular polyoxyalkylene polyols.

Most preferred are polyoxypropylene di- or triols or ethylene oxide-terminated polyoxypropylene di- or triols.

Preference is given to polyols having an average molecular weight Mₙ within a range from 400 to 20 000 g/mol, preferably from 1000 to 15 000 g/mol.

Preference is given to polyols having an average OH functionality within a range from 1.6 to 3.

Preference is given to polyols that are liquid at room temperature.

For the production of a polymer containing isocyanate groups, it is also possible to additionally use fractions of di- or polyfunctional alcohols, in particular ethane-1,2-diol, propane-1,2-diol, propane-1,3-diol, 2-methylpropane-1,3-diol, butane-1,2-diol, butane-1,3-diol, butane-1,4-diol, pentane-1,3-diol, pentane-1,5-diol, 3-methylpentane-1,5-diol, neopentyl glycol, dibromoneopentyl glycol, hexane-1,2-diol, hexane-1,6-diol, heptane-1,7-diol, octane-1,2-diol, octane-1,8-diol, 2-ethylhexane-1,3-diol, diethylene glycol, triethylene glycol, dipropylene glycol, tripropylene glycol, cyclohexane-1,3-dimethanol or -1,4-dimethanol, ethoxylated bisphenol A, propoxylated bisphenol A, cyclohexanediol, hydrogenated bisphenol A, dimer fatty acid alcohols, 1,1,1-trimethylolethane, 1,1,1-trimethylolpropane, glycerol, pentaerythritol, sugar alcohols, such as in particular xylitol, sorbitol or mannitol, or sugars, such as in particular sucrose, or alkoxylated derivatives of the alcohols mentioned or mixtures of the alcohols mentioned.

The moisture-curing polyurethane composition preferably comprises at least one polymer containing isocyanate groups.

The polymer containing isocyanate groups preferably has an average molecular weight Mₙ within a range from 1500 to 20 000 g/mol, in particular 2000 to 15 000 g/mol.

The polymer containing isocyanate groups preferably has a content of isocyanate groups within a range from 0.5% to 10% by weight, in particular 1% to 5% by weight.

The polymer containing isocyanate groups preferably has a low content of monomeric diisocyanate, preferably of less than 2% by weight, in particular less than 1% by weight of monomeric diisocyanate.

The moisture-curing polyurethane composition preferably additionally comprises at least one further constituent selected from fillers, plasticizers, further blocked amines, catalysts, and stabilizers.

Suitable fillers are in particular ground or precipitated calcium carbonates, optionally coated with fatty acids, in particular stearates, barytes, quartz flours, quartz sands, dolomites, wollastonites, calcined kaolins, sheet silicates, such as mica or talc, zeolites, aluminum hydroxides, magnesium hydroxides, silicas, including finely divided silicas from pyrolysis processes, cements, gypsums, fly ashes, industrially produced carbon blacks, graphite, metal powders, for example of aluminum, copper, iron, silver or steel, PVC powders or lightweight fillers such as hollow glass beads or gas-filled plastic spheres (microspheres), in particular the types obtainable under the Expancel® brand name (from Akzo Nobel).

Preference is given to calcium carbonates that have optionally been coated with fatty acids, in particular stearates, calcined kaolins, finely divided silicas or industrially produced carbon blacks.

Suitable plasticizers are in particular carboxylic esters, such as phthalates, in particular diisononyl phthalate (DINP), diisodecyl phthalate (DIDP) or di(2-propylheptyl)phthalate (DPHP), hydrogenated phthalates or cyclohexane-1,2-dicarboxylate esters, in particular hydrogenated diisononyl phthalate or diisononyl cyclohexane-1,2-dicarboxylate (DINCH), terephthalates, in particular bis(2-ethylhexyl) terephthalate (DOTP) or diisononyl terephthalate (DINT), hydrogenated terephthalates or cyclohexane-1,4-dicarboxylate esters, in particular hydrogenated bis(2-ethylhexyl) terephthalate or bis(2-ethylhexyl) cyclohexane-1,4-dicarboxylate, or hydrogenated diisononyl terephthalate or diisononyl cyclohexane-1,4-dicarboxylate, isophthalates, trimellitates, adipates, in particular dioctyl adipate, azelates, sebacates, benzoates, glycol ethers, glycol esters, plasticizers having polyether structure, in particular polypropylene oxide monools, diols or triols having blocked hydroxyl groups, in particular in the form of acetate groups, organic phosphoric or sulfonic esters, polybutenes, polyisobutenes or plasticizers derived from natural fats or oils, in particular epoxidized soybean or linseed oil.

Preferred plasticizers are phthalates, hydrogenated phthalates, adipates or plasticizers having polyether structure.

Suitable further blocked amines are in particular oxazolidines or aldimines. Preferred as a further blocked amine is a bisoxazolidine of the formula (VIII) or (IX),

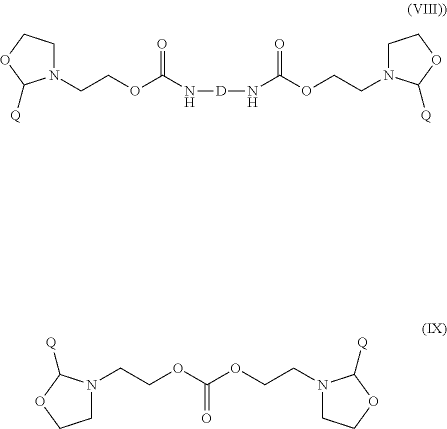

where
D is a divalent hydrocarbyl radical having 6 to 15 carbon atoms, in particular 1,6-hexylene or (1,5,5-trimethylcyclohexan-1-yl)methane-1,3 or 4(2)-methyl-1,3-phenylene, and
Q is a monovalent organic radical having 3 to 26 carbon atoms, in particular 2-propyl, 3-heptyl, phenyl or a substituted phenyl radical, in particular a phenyl radical substituted in the para position with an optionally branched decylphenyl, undecylphenyl, dodecylphenyl, tridecylphenyl or tetradecylphenyl radical.

Also preferred as a further blocked amine is a monooxazolidine of the formula

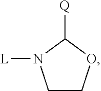

where L is an alkyl, cycloalkyl or arylalkyl radical having 1 to 8 carbon atoms, in particular methyl, ethyl or n-butyl, and Q is as defined previously.

Also preferred as a further blocked amine is an aldimine of the formula G [symbol] ═B]_{y}, where y is 2 or 3, G is an organic radical having 2 to 23 carbon atoms, and B is an organic radical having 6 to 30 carbon atoms.

G is preferably an alkylene radical optionally having cyclic components or a di- or trivalent polyoxyalkylene radical having 5 to 15 carbon atoms, in particular 1,6-hexylene, (1,5,5-trimethylcyclohexan-1-yl)methane-1,3 or α,ω-polyoxypropylene having an average molecular weight Mₙ within a range from 170 to 300 g/mol or trimethylolpropane-started tris(ω-polyoxypropylene) having an average molecular weight Mₙ within a range from 330 to 500 g/mol.

B is preferably an organic radical having 7 to 22 carbon atoms, in particular 2,2-dimethyl-3-(N-morpholino)propylidene, 2,2-dimethyl-3-lauroyloxypropylidene, benzylidene or substituted benzylidene, in particular 4-decylbenzylidene, 4-undecylbenzylidene, 4-dodecylbenzylidene, 4-tridecylbenzylidene or 4-tetradecylbenzylidene, in which the 4-alkyl radicals are optionally branched.

The moisture-curing polyurethane composition particularly preferably comprises at least one bisoxazolidine of the formula (VIII), in which D is 1,6-hexylene. Such a composition affords particularly high strengths coupled with high extensibility.

Suitable catalysts are catalysts for accelerating the reaction of isocyanate groups, in particular organotin(IV) compounds, such as in particular dibutyltin diacetate, dibutyltin dilaurate, dibutyltin dichloride, dibutyltin diacetylacetonate, dimethyltin dilaurate, dioctyltin diacetate, dioctyltin dilaurate or dioctyltin diacetylacetonate, complexes of bismuth(III) or zirconium(IV), in particular with ligands selected from alkoxides, carboxylates, 1,3-diketonates, oxinate, 1,3-ketoesterates, and 1,3-ketoamidates, or compounds containing tertiary amino groups, such as in particular 2,2′-dimorpholinodiethyl ether (DMDEE).

Suitable catalysts are additionally catalysts for the hydrolysis of aldimine groups, in particular organic acids, in particular carboxylic acids, such as 2-ethylhexanoic acid, lauric acid, stearic acid, isostearic acid, oleic acid, neodecanoic acid, benzoic acid, salicylic acid or 2-nitrobenzoic acid, organic carboxylic anhydrides, such as phthalic anhydride, hexahydrophthalic anhydride or hexahydromethylphthalic anhydride, silyl esters of carboxylic acids, organic sulfonic acids, such as methanesulfonic acid, p-toluenesulfonic acid or 4-dodecylbenzenesulfonic acid, sulfonic esters, other organic or inorganic acids, or mixtures of the abovementioned acids and acid esters. Particular preference is given to carboxylic acids, in particular aromatic carboxylic acids, such as benzoic acid, 2-nitrobenzoic acid or in particular salicylic acid.

Also especially suitable are combinations of different catalysts.

Suitable stabilizers are in particular stabilizers against oxidation, heat, light or UV radiation, in particular titanium dioxides, iron oxides, zinc oxides, benzophenones, benzotriazoles, compounds having 2,6-di-tert-butylphenol groups, as known for example under the Irganox® trade name (from BASF), compounds having 2,2,6,6-tetramethylpiperidine groups, called HALS (hindered amine light stabilizers), as known for example under the Tinuvin® trade name (from BASF), or phosphorus-containing compounds as known for example under the Irgafos® trade name (from BASF).

The moisture-curing polyurethane composition may contain further additions, in particular
inorganic or organic pigments, in particular titanium dioxide, chromium oxides or iron oxides;
fibers, in particular glass fibers, carbon fibers, metal fibers, ceramic fibers, polymer fibers, such as polyamide fibers or polyethylene fibers, or natural fibers, such as wool, cellulose, hemp or sisal;
nanofillers such as graphene or carbon nanotubes;
dyes;
desiccants, in particular molecular sieve powders, calcium oxide, highly reactive isocyanates such as p-tosyl isocyanate, monooxazolidines such as Incozol® 2 (from Incorez) or orthoformic esters;
adhesion promoters, in particular organoalkoxysilanes, in particular epoxysilanes, such as in particular 3-glycidoxypropyltrimethoxysilane or 3-glycidoxypropyltriethoxysilane, (meth)acrylosilanes, anhydridosilanes, carbamatosilanes, alkylsilanes or iminosilanes, or oligomeric forms of these silanes, or titanates;
further catalysts that accelerate the reaction of the isocyanate groups;
rheology modifiers, in particular thickeners, in particular sheet silicates, such as bentonites, derivatives of castor oil, hydrogenated castor oil, polyamides, polyamide waxes, polyurethanes, urea compounds, fumed silicas, cellulose ethers or hydrophobically modified polyoxyethylenes;
solvents, in particular acetone, methyl acetate, tert-butyl acetate, 1-methoxy-2-propyl acetate, ethyl 3-ethoxypropionate, diisopropyl ether, diethylene glycol diethyl ether, ethylene glycol diethyl ether, ethylene glycol monobutyl ether, ethylene glycol mono-2-ethylhexyl ether, acetals such as propylal, butylal, 2-ethylhexylal, dioxolane, glycerol formal or 2,5,7,10-tetraoxaundecane (TOU), toluene, xylene, heptane, octane, naphtha, white spirit, petroleum ether or gasoline, in particular Solvesso™ grades (from Exxon), and propylene carbonate, dimethyl carbonate, butyrolactone, N-methylpyrrolidone, N-ethylpyrrolidone, p-chlorobenzotrifluoride or benzotrifluoride;
natural resins, fats or oils, such as rosin, shellac, linseed oil, castor oil or soybean oil;
nonreactive polymers, in particular homo- or copolymers of unsaturated monomers, in particular from the group comprising ethylene, propylene, butylene, isobutylene, isoprene, vinyl acetate or alkyl (meth)acrylates, in particular polyethylenes (PE), polypropylenes (PP), polyisobutylenes, ethylene/vinyl acetate copolymers (EVA) or atactic poly-α-olefins (APAO);
flame-retardant substances, in particular the aluminum hydroxide or magnesium hydroxide fillers already mentioned, and also in particular organic phosphoric esters, such as in particular triethyl phosphate, tricresyl phosphate, triphenyl phosphate, diphenyl cresyl phosphate, isodecyl diphenyl phosphate, tris(1,3-dichloro-2-propyl) phosphate, tris(2-chloroethyl) phosphate, tris(2-ethylhexyl) phosphate, tris(chloroisopropyl) phosphate, tris(chloropropyl) phosphate, isopropylated triphenyl phosphate, mono-, bis- or tris(isopropylphenyl) phosphates having varying degrees of isopropylation, resorcinol bis(diphenylphosphate), bisphenol A bis(diphenylphosphate) or ammonium polyphosphates;
additives, in particular wetting agents, leveling agents, defoamers, deaerating agents or biocides;
   or further substances customarily used in moisture-curing polyurethane compositions.

It may be advisable to chemically or physically dry certain substances before mixing them into the composition.

The moisture-curing polyurethane composition is in particular produced with exclusion of moisture and stored at ambient temperature in moisture-tight containers. A suitable moisture-tight container is made in particular from an optionally coated metal and/or plastic, and is in particular a drum, a container, a hobbock, a bucket, a canister, a can, a bag, a tubular bag, a cartridge or a tube.

The moisture-curing polyurethane composition may be in the form of a one-component composition or in the form of a multi-component, in particular two-component, composition.

A composition referred to as a “one-component” composition is one in which all constituents of the composition are in the same container and which is storage-stable as is.

A composition referred to as a “two-component” composition is one in which the constituents of the composition are present in two different components that are stored in separate containers and are not mixed with one another until shortly before or during the application of the composition.

The moisture-curing polyurethane composition is preferably a one-component composition. Given suitable packaging and storage, it is storage-stable, typically for several months up to one year or longer.

On application of the moisture-curing polyurethane composition, the curing process commences. This results in the cured composition.

In the case of a one-component composition, it is applied as is and then begins to cure under the influence of moisture or water. To accelerate curing, an accelerator component containing water and optionally a catalyst and/or a curing agent can be mixed into the composition on application, or the composition, once it has been applied, can be contacted with such an accelerator component.

On curing, the isocyanate groups react under the influence of moisture with the hydrolyzing aldimine groups and further blocked amino groups optionally present and—in parallel thereto or subsequently—also with one another to form urea groups. The totality of these and any other reactions of isocyanate groups that lead to curing of the composition is also referred to as crosslinking.

The moisture needed for curing the moisture-curing polyurethane composition preferably gets into the composition through diffusion from the air (atmospheric moisture). This process results in the formation of a solid layer of cured composition (skin) on the surfaces of the composition in contact with air. Curing proceeds in the direction of diffusion from the outside inward, the skin becoming increasingly thick and ultimately covering the entire composition that was applied. The moisture can also get into the composition additionally or entirely from one or more substrate(s) to which the composition has been applied and/or can come from an accelerator component that is mixed into the composition on application or is contacted therewith after application, for example by painting or spraying.

The moisture-curing polyurethane composition is preferably applied at ambient temperature, in particular within a range from about −10 to 50° C., preferably within a range from −5 to 45° C., in particular 0 to 40° C.

Curing of the moisture-curing polyurethane composition takes place preferably at ambient temperature.

Preference is given to using the moisture-curing polyurethane composition as adhesive or sealant or coating in particular in the construction and manufacturing industries or in motor vehicle construction.

Preference is given to use as elastic adhesive and/or sealant, in particular for parquet bonding, assembly, bonding of installable components, module bonding, pane bonding, join sealing, bodywork sealing, seam sealing or cavity sealing or for elastic bonds in motor vehicle construction, such as in particular the bonded attachment of parts such as plastic covers, trim strips, flanges, fenders, driver's cabins or other installable components to the painted body of a motor vehicle, or the bonding of panes into the vehicle body, said motor vehicles in particular being automobiles, trucks, buses, rail vehicles or ships.

Also preferred is use as elastic coating for protection of floors or walls, in particular as a so-called liquid-applied membrane for sealing of roofs, in particular flat roofs or slightly inclined roof areas or gardens, or in building interiors for water sealing, for example beneath tiles or ceramic slabs in wet rooms or kitchens or on balconies, or as seam seal, or for repair purposes as seal or coating, for example of leaking roof membranes or other elastic seals.

### EXAMPLES

Working examples are presented hereinbelow, the purpose of which is to further elucidate the described invention. The invention is of course not limited to these described working examples.

“Standard climatic conditions” (“SCC”) refer to a temperature of 23±1° C. and a relative air humidity of 50±5%.

Unless otherwise stated, the chemicals used were from Sigma-Aldrich Chemie GmbH.

Description of the Measurement Methods:

Gas chromatograms (GC) were measured within a temperature range from 60 to 320° C. at a heating rate of 15° C./min and a 10 min hold time at 320° C. The injector temperature was 250° C. A Zebron ZB-5 column was used (L=30 m, ID=0.25 mm, dj=0.5 μm) at a gas flow of 1.5 ml/min. Detection was by flame ionization (FID). For assignment of GC peaks to chemical structures, a mass spectrum (EI⁺) was additionally recorded.

Infrared spectra (FT-IR) were recorded as neat films on a Bruker Alpha Eco-ATR FT-IR instrument. Absorption bands are reported in wavenumbers (cm⁻¹).

DSC (differential scanning calorimetry) analyses were determined on a Mettler Toledo DSC 3+ 700 instrument in a temperature range of 10 to 400° C. with a heating rate of 4 K/m in using adiabatic M20 pressure crucibles (from TÛV Sûd, Switzerland) (first run).

The amine value (including blocked amino groups) was determined by titration (with 0.1N HClO₄ in acetic acid against crystal violet).

Preparation of Aldol Esters of the Formula (I):

### Example 1

### Preparation of the Inventive Reaction Product Comprising 2,2-dimethyl-3-acetoxypropanal in the Presence of Triethylamine

Step 1 (Aldol Reaction):

A V4A steel reactor equipped with addition, stirring, heating, and cooling system and a distillation column with condenser and maintained under an atmosphere of nitrogen was charged with 297 kg of triethylamine (from BASF), 587 kg of paraformaldehyde (from Tennants Fine Chemicals), and 282 kg of deionized water and this was mixed. The mixture was heated under reflux to 60° C. with stirring. Into this was then metered 1523 kg isobutyraldehyde (from BASF) over a period of 3 hours, during which the reaction mixture was maintained under reflux at 65 to 75° C. After a further 30 min at reflux, no more exothermicity was discernible. The system was then switched over to distillation, the internal pressure was gradually reduced, and the volatiles were distilled off, firstly at 85° C./250 mbar and then at 100° C./50 mbar. 705 kg of distillate was collected (which according to gas chromatography was unreacted isobutyraldehyde, water, and a substantial part of the triethylamine). Remaining in the reactor was 1924 kg of reaction mixture, which according to gas chromatography comprised approx. 88% by weight of 2,2-dimethyl-3-hydroxypropanal (retention time approx. 3.2 min) and approx. 4% by weight of triethylamine (retention time 2.2 min).

Step 2 (Esterification):

The reactor was then brought to standard pressure with nitrogen, brought to reflux, and the internal temperature increased to 110° C. The internal pressure was then reduced to 250 mbar and 2076 kg of acetic anhydride (from BP Chemicals) added and mixed in over a period of 1 hour. This was then followed by removal of volatiles from the reaction mixture. For this, the reactor was set to fractional distillation (80% reflux) and the contents distilled at an overhead temperature of approx. 78° C. As soon as the overhead temperature reached 80° C., the internal pressure in the reactor was gradually reduced further and distillation each time continued until the overhead temperature again reached 80° C. Once the overhead temperature had exceeded 80° C. at an internal pressure of 30 mbar, the distillation, i.e. the removal of volatiles from the reaction mixture, was ended. A total of 2134 kg of distillate was collected (which according to gas chromatography was unreacted acetic anhydride, acetic acid, triethylamine, and 2,2-dimethyl-3-acetoxypropanal). The reaction product was then cooled and maintained under a nitrogen atmosphere.

1851 kg of a clear, pale yellowish liquid with a mildly fruity odor was obtained. The reaction product comprised according to gas chromatography approx. 78% by weight of 2,2-dimethyl-3-acetoxypropanal (retention time 4.8 min), approx. 5.7% by weight of triesters of the formula (V) (retention time 10.9 min), and approx. 6.3% by weight of acetal of the formula (VI) (retention time 6.4 min and 6.6 min). This is hereinafter referred to as “reaction product from example 1”.

FT-IR: 2973, 2938, 2877, 2818, 2716, 1728, 1473, 1374, 1228, 1160, 1118, 1040, 892, 775.

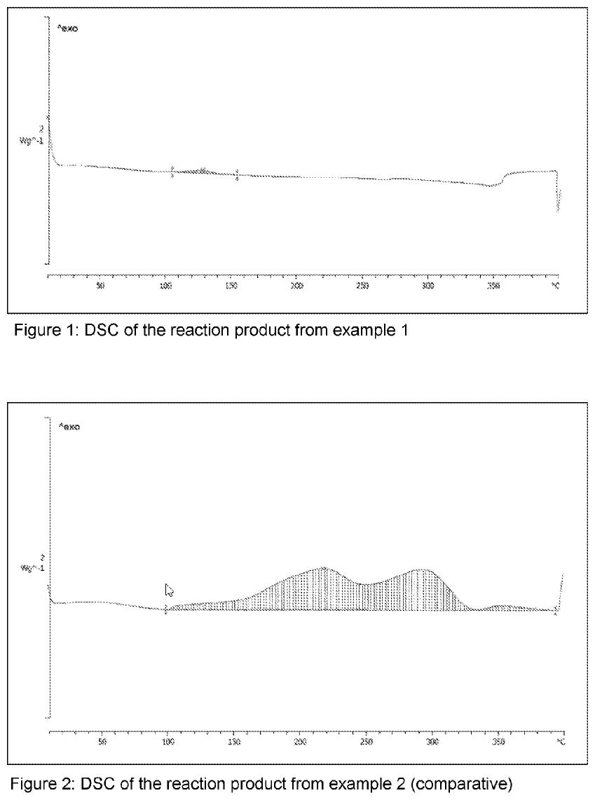

A DSC of the reaction product was recorded, which is shown in FIG. 1. Weak exothermicity of 20 kJ/kg in the region from 105 to 155° C. was determined.

Purification of the reaction product by overhead distillation: (as comparison) 500 g of the reaction product obtained from example 1 was distilled under reduced pressure at 120 to 130° C. in a round-bottomed flask with distillation column. This yielded 370.4 g of distillate (=overhead-distilled 2,2-dimethyl-3-acetoxypropanal from example 1) at an overhead temperature of 84 to 87° C., 30 mbar and 60% reflux, which according to gas chromatography comprised approx. 94% by weight of 2,2-dimethyl-3-acetoxypropanal.

The first fraction (=first runnings) of 73.8 g was collected at an overhead temperature of 76 to 80° C., 30 mbar, and 80% reflux. This comprised according to gas chromatography approx. 56% by weight of 2,2-dimethyl-3-acetoxypropanal, approx. 17% by weight of acetic acid, and approx. 18% by weight of triethylamine. Left behind as a residue was 55.8 g having a content of 2,2-dimethyl-3-acetoxypropanal of 0.8% by weight.

### Example 2: (Comparative)

### Preparation of 2,2-dimethyl-3-acetoxypropanal in the Presence of Acid

A round-bottomed flask with distillation column and water separator was charged under a nitrogen atmosphere with 100 g of cyclohexane, 144.0 g of paraformaldehyde, 403.7 g of acetic acid, and 6.3 g of p-toluenesulfonic acid and mixed. The mixture was heated under reflux to 60° C. with thorough stirring and to this was slowly added 346.4 g of isobutyraldehyde such that the internal temperature did not rise above 75° C. The system was then switched from reflux to water separation and heated gradually to an internal temperature of 100° C. Once the internal temperature had reached 100° C., the internal pressure was gradually reduced, making sure that the internal temperature was maintained at about 100° C. At an internal pressure of 600 mbar, 81 g of water was separated. The system was then switched from water separation to distillation and the internal pressure reduced further, such that the internal temperature was maintained at about 100° C. At an internal pressure of 30 mbar and an overhead temperature of 67° C., the excess acetic acid was mostly removed. The reaction product was cooled and maintained under a nitrogen atmosphere. The distillate collected consisted according to gas chromatography mostly of cyclohexane, a little water, isobutyraldehyde, and acetic acid.

576 g of a dark-colored liquid with a pungent odor was obtained. The reaction product comprised according to gas chromatography approx. 61.7% by weight of 2,2-dimethyl-3-acetoxypropanal (retention time 4.8 min).

A DSC of the reaction product from example 2 was recorded, which is shown in FIG. 2. Strong exothermicity of 530 kJ/kg in the region from 100 to 400° C. was determined.

Preparation of Blocked Amines:

Aldimine A1: (from the Inventive Reaction Product)

### N,N′-Bis(2,2-dimethyl-3-acetoxypropylidene)-3-aminomethyl-3,5,5-trimethylcyclohexylamine

A round-bottomed flask was charged under an atmosphere of nitrogen with 373.0 g of the reaction product from example 1 comprising approx. 78% by weight of 2,2-dimethyl-3-acetoxypropanal. To this was then added with thorough stirring 170.3 g (1 mol) of 3-aminomethyl-3,5,5-trimethylcyclohexylamine (Vestamin® IPD, from Evonik), after which volatiles were removed at 80° C. and a vacuum of 10 mbar. This yielded 497 g of a clear, pale yellowish, low-viscosity liquid with a mildly fruity odor and an amine value of 223 mg KOH/g, which corresponds to a calculated aldimine equivalent weight of 252 g/equiv.

Aldimine R1: (Comparison, from Purified Reaction Product)

N,N′-Bis(2,2-dimethyl-3-acetoxypropylidene)-3-aminomethyl-3,5,5-trimethylcyclohexylamine

A round-bottomed flask was charged under an atmosphere of nitrogen with 293 g of overhead-distilled 2,2-dimethyl-3-acetoxypropanal from example 1. To this was then added with thorough stirring 170.3 g (1 mol) of 3-aminomethyl-3,5,5-trimethylcyclohexylamine (Vestamin® IPD, from Evonik), after which volatiles were removed at 80° C. and a vacuum of 10 mbar. This yielded 418 g of a clear, almost colorless, low-viscosity liquid with a mildly fruity odor and an amine value of 262 mg KOH/g, which corresponds to a calculated aldimine equivalent weight of 214 g/equiv.

Moisture-Curing Polyurethane Compositions:

Compositions Z1 and Z2

For each composition, the following constituents were mixed in a centrifugal mixer with the exclusion of moisture until a macroscopically homogeneous liquid had formed:

213.7 g of a polymer containing isocyanate groups and having an NCO content of 3.7% by weight, based on a polyoxypropylenediol having an OH value of 56 mg KOH/g and toluene diisocyanate (Desmodur® T 80 P, from Covestro), 61.3 g of crosslinker (Desmodur® L67 MPA/X, from Covestro), 73 g of plasticizer, 149 g of solvent, 19 g of thickener, 417 g of inorganic filler, and 0.5 g of salicylic acid. To this was additionally added 67.7 g of aldimine A1 in the case of composition Z1 or 57.5 g of aldimine A1 in the case of composition Z2.

Each composition was stored in a tightly closed metal container with the exclusion of moisture and finally tested as follows:

The viscosity was determined using a Rotothinner at 20° C.: “freshly” refers to the measured viscosity 24 h after production of the composition. “4 w 40° C.” and “8 w 40° C.” refers to the viscosity after storage for respectively 4 weeks and 8 weeks at 40° C. in closed containers.

The curing rate (“BK drying time”) was determined under standard climatic conditions using a Beck-Koller drying time recorder in accordance with ASTM D5895. The results for phase 2 correspond to the skin-over time (tack-free time) of the composition.

Through-curing was determined by applying the composition in the form of a cylinder of 40 mm diameter and 4 mm height, allowing it to stand in standard climatic conditions (SCC) or at 5° C./80% relative humidity, cutting this open after 24 h or 48 h, and measuring the thickness of the cured layer that had formed on the surface of the composition. The results are reported as “24 h SCC” and “48 h SCC” and “48 h 5° C.”, according to the curing time and climatic conditions. For determination of the mechanical properties, a two-layer cured film was produced for each composition. This was done by applying a first layer in a thickness of 800 μm with a doctor blade and storing for 24 h in standard climatic conditions, followed by a second layer applied with a doctor blade in a thickness of 400 μm at an angle of 90° relative to the first layer. This two-layer film was stored in standard climatic conditions for a further 24 h, followed by 24 h in an air-circulation oven at 60° C. After a further 24 h in standard climatic conditions, strip-shaped test specimens of 100 mm length and 25 mm width were punched out of the film and used to determine the tensile strength and elongation at break in accordance with DIN EN 53504 at a strain rate of 180 mm/min and with a track length of 60 mm.

The appearance was determined optically on the film produced for the determination of mechanical properties.

The odor was determined by smelling through the nose, at a distance of about 100 mm, a freshly applied flat composition of about 150 mm diameter.

TABLE 1
Properties of compositions Z1 and Z2.

| Composition | | Z1 | Z2 (comparison) |
|---|---|---|---|
| Viscosity [mPa · s] | freshly | 1800 | 1950 |
| | 4 weeks 40° C. | 2200 | 2400 |
| | 8 weeks 40° C. | 2350 | 2500 |
| BK drying time | phase 2 | 1:38 | 1:30 |
| [h:min] | phase 4 | 2:53 | 3:00 |
| Through-curing | 24 h SCC | 2.6 | 2.6 |
| (mm depth) | 48 h SCC | 3.9 | 4.0 |
| | 48 h 5° C. | 3.8 | 3.8 |

| | | |
|---|---|---|
| Tensile strength [MPa] | 5.59 | 5.50 |
| Elongation at break [%] | 328 | 260 |
| Appearance | matt, nontacky, | matt, nontacky, |
| | no bubbles | no bubbles |
| Odor | mild, solvent- | mild, solvent- |
| | like, slightly | like, slightly |
| | fruity | fruity |

It can be seen from Table 1 that the inventive reaction product from example 1 is of excellent suitability as is, i.e. without further purification by overhead distillation, for the preparation of aldimine A1, which is used as a blocked amine/latent curing agent in a one-component moisture-curing composition. Composition Z1 in some cases surprisingly even exhibits better properties than composition Z2, which comprises aldimine R1 derived from 2,2-dimethyl-3-acetoxypropanal purified by overhead distillation. In particular, composition Z1 shows especially lower viscosity, even after storage, and especially high elongation, remaining properties being otherwise comparable.

Compositions Z1 and Z2 are suitable in particular as coating or covering, in particular as so-called liquid applied membrane for the sealing of roofs, bridges, terraces, etc.

1. A method for preparing an aldol ester of the formula (I), 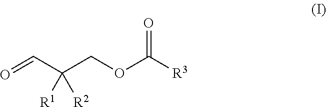
where R¹ and R² are identical or different alkyl radicals having 1 to 4 carbon atoms or together are an alkylene radical having 4 to 6 carbon atoms, and R³ is an optionally halogenated hydrocarbyl radical having 1 to 17 carbon atoms,
wherein at least one carboxylic anhydride of the formula (II) 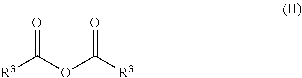
is reacted with at least one aldol of the formula (III), 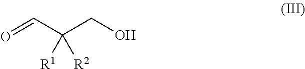
optionally in the form of an oligomer thereof, while heating in the presence of a basic catalyst having a conjugate acid pKa of at least 8.

2. The method as claimed in claim 1, wherein R¹ and R² are each methyl.

3. The method as claimed claim 1, wherein R³ is an alkyl radical having 1 to 7 carbon atoms or is phenyl.

4. The method as claimed in claim 1, wherein the basic catalyst has a conjugate acid pKa of at least 9.

5. The method as claimed in claim 1, wherein the basic catalyst is triethylamine.

6. The method as claimed in claim 1, wherein it is executed at a temperature within a range from 80 to 150° C.

7. The method as claimed in claim 1, wherein it is executed without using an organic solvent or entraining agent.

8. The method as claimed in claim 1, wherein the aldol of the formula (III) is used as constituent of a reaction mixture obtained from the reaction of formaldehyde, optionally in the form of paraformaldehyde or trioxane, with at least one aldehyde of the formula (IV) 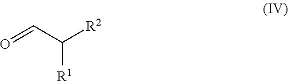
in the presence of a basic catalyst having a conjugate acid pKa of at least 8.

9. The method as claimed in claim 1, wherein it is executed in two stages, wherein
(i) in the first step, the basic catalyst and formaldehyde are initially charged, then at least one aldehyde of the formula (IV) 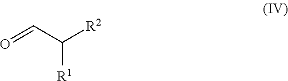
is added in stoichiometric excess in relation to formaldehyde at a temperature within a range from 60 to 90° C., resulting in the formation of the aldol of the formula (III), after which volatiles are removed from the reaction mixture, and
(ii) in the second step, the reaction mixture thus obtained is reacted with the carboxylic anhydride of the formula (II) at a temperature within a range from 100 to 130° C., wherein volatiles are removed from the reaction mixture during and/or after the reaction.

10. A reaction product obtained from the method as claimed in claim 1, wherein it comprises 60% to 95% by weight of aldol ester of the formula (I) and 5% to 40% by weight of other esters, aldehydes and/or acetals not corresponding to the formula (I).

11. The reaction product as claimed in claim 10, wherein it comprises triesters of the formula (V) and/or acetals of the formula (VI),

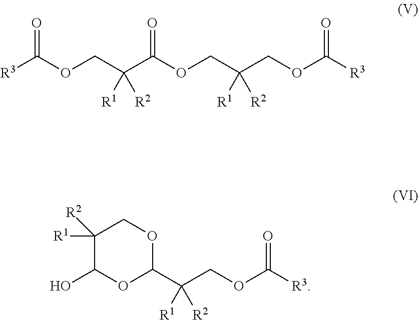

12. A blocked amine obtained from reacting the reaction product as claimed in claim 10 with at least one amine that has a primary amino group and additionally at least one reactive group selected from primary amino group, secondary amino group, and hydroxyl group.

13. The blocked amine as claimed in claim 12, wherein the amine is selected from the group consisting of hexane-1,6-diamine, 1-amino-3-aminomethyl-3,5,5-trimethylcyclohexane, 4(2)-methylcyclohexane-1,3-diamine, 1,3-bis(aminomethyl)cyclohexane, 1,4-bis(aminomethyl)cyclohexane, 1,3-bis(aminomethyl)benzene, cyclohexane-1,2-diamine, cyclohexane-1,3-diamine, cyclohexane-1,4-diamine, bis(4-aminocyclohexyl)methane, 2,5(2,6)-bis(aminomethyl)bicyclo[2.2.1]heptane, 3(4),8(9)-bis(aminomethyl)tricyclo[5.2.1.0^{2,6}]decane, α,ω-polyoxypropylenediamine having an average molecular weight Mn within a range from 170 to 500 g/mol, trimethylolpropane- or glycerol-started tris(ω-polyoxypropylenamine) having an average molecular weight Mn within a range from 330 to 500 g/mol, 1,4-phenylenediamine, 3,5-diethyl-2,4(6)-tolylenediamine, 2-(2-aminoethoxy)ethanol, 2-(2-(2-aminoethoxy)ethoxy)ethanol, and 3-aminomethyl-3,5,5-trimethylcyclohexanol.

14. A polyurethane composition comprising
at least one polyisocyanate and/or polymer containing isocyanate groups and
at least one blocked amine as claimed in claim 12.

15. A method comprising using the polyurethane composition as claimed in claim 14 as adhesive or sealant or coating.
